(19) United States
(12) Patent Application Publication
(10) Pub. No.: US 2008/0076130 A1
(43) Pub. Date: Mar. 27, 2008

(54) MOLECULAR HAPLOTYPING OF GENOMIC DNA

(75) Inventor: Baochuan Guo, Solon, OH (US)

Correspondence Address: CALFEE HALTER & GRISWOLD, LLP, 800 SUPERIOR AVENUE, SUITE 1400, CLEVELAND, OH, 44114, US

(73) Assignee: CLEVELAND STATE UNIVERSITY

(21) Appl. No.: 11/780,351

(22) Filed: Jul. 19, 2007

Related U.S. Application Data
(63) Continuation of application No. 10/798,718, filed on Mar. 11, 2004.
(60) Provisional application No. 60/453,516, filed on Mar. 12, 2003.

(51) Int. Cl. C12Q 1/68; G01N 33/48

(52) U.S. Cl. 435/6; 436/94

(57) ABSTRACT

A method of determining the haplotype structures of a nucleic acid comprising two or more single nucleotide polymorphisms (SNPs) of interest is provided. The method comprises obtaining an enriched nucleic acid fraction which comprises from 2 to 30 times more of one allelic variant of the nucleic acid than the other allelic variant of the nucleic acid, and genotyping the enriched nucleic acid fraction to identify the alleles in the two or more SNPs of interest that are present at a higher level or lower level than the other alleles in said two or more SNPs of interest, wherein the alleles that are present at a higher level are on the enriched allelic variant and form one haplotype and the alleles that are present at a lower level are on the non-enriched allelic variant and form the other haplotype. Kits for conducting the present methods are also provided.

### CROSS-REFERENCE TO RELATED APPLICATION

This invention claims priority to U.S. Provisional Patent Application Ser. No. 60/453,516 filed Mar. 12, 2003, which is incorporated herein in its entirety.

### STATEMENT ON GOVERNMENT FUNDED RESEARCH

This invention was made, at least in part, with government support under National Institutes of Health Grant No. HG01815 and CA-81653. The U.S. government has certain rights in the invention.

### FIELD OF THE INVENTION

The present invention is related to methods of determining the haplotype structure of nucleic acid comprising two or more single nucleotide polymorphisms, particularly genomic DNA fragments in which at least two of the single nucleotide polymorphisms are separated by five or more kilobases.

### BACKGROUND OF THE INVENTION

A “single nucleotide polymorphism” or “SNP” is a single base pair (i.e., a pair of complementary nucleotide residues on opposite genomic strands) within a DNA region wherein the identities of the paired nucleotide residues vary from individual to individual. When two or more SNPs occur within a particular region of genomic DNA, each allele of the genomic DNA region is known as a “haplotype.” It is often useful to identify the haplotypes in an individual, for example, to appropriately diagnose a condition of the individual.

Investigators have identified millions of nucleotide positions where single base changes, base insertions, or base deletions may occur in the human genome. These genetic variations in the genetic composition of an individual determine genetic diseases, predisposition to diseases, ability to metabolize therapeutics, rate of metabolism of therapeutics, side effects of therapeutics, and the like.

Typically, in samples of DNA or cDNA derived from tissues or cells that have two chromosomes (i.e., all normal somatic tissues in humans and animals) in which there are two or more heterozygous sites, it is generally impossible to tell which nucleotides belong together on one chromosome when using genotyping methods such as (i) DNA sequencing, (ii) nucleic acid hybridization of oligonucleotides to genomic DNA or total cDNA or amplification products derived therefrom, (iii) nucleic acid hybridization using probes derived from genomic DNA or total cDNA or amplification products derived therefrom, or (iv) most amplification-based schemes for variance detection.

Recently, an international research consortium launched a public-private effort to create the next generation map of the human genome [1]. Called the International HapMap Project, this new venture is aimed at speeding the discovery of genes related to common illnesses such as asthma, cancer, diabetes and heart disease. The HapMap will find the blocks into which the genome is organized, each of which may contain dozens of SNPs. Because of the block pattern of haplotypes, it will be possible to identify just a few SNP variants in each block to uniquely mark, or tag that haplotype. As a result, researches will need to study only about 300,000 to 600,000 tag SNPs to identify the haplotypes in the human genome. Researchers then only need to detect a few tag SNPs to identify that unique block of genome and to know all of the SNPs associated with that one block. This strategy works because SNP variants that lie close to each other along DNA form a haplotype block and tend to be inherited together. SNP variants that are far from each other along DNA tend to be in different haplotype blocks and are less likely to be inherited together [2,3].

Once the HapMap is constructed, it can be used to study the genetic risk factors underlying a wide range of diseases and conditions. For any given disease, researchers would use the HapMap tag SNPs to compare the haplotype patterns of a group of people known to have the disease to a group of people without the disease. If the association study finds a certain haplotype more often in the people with the disease, one would then zero in on that genomic region in their search for the specific genetic variant. The tag SNPs would serve as signposts indicating that a genetic variant involved in the disease may lie nearby.

Mapping an individual's haplotypes also may be used in the future to help customize medical treatment. Genetic variation has been shown to affect the response of patients to drugs, toxic substances and other environmental factors. Some already envision an area in which drug treatment is customized, based on the patient's haplotypes, to maximize the effectiveness of the drug while minimizing side effects. In addition, the HapMap may eventually help pinpoint genetic variations that may contribute to good health, such as those protecting against infectious diseases or promoting longevity.

Carrying out such a complex project depends on the application of robust technologies to analyze individual haplotypes [4]. Haplotyping is a process of determining the specific pattern of particular SNPs on one of an individuals chromosomes. They are (1) reconstruction of the haplotypes of sampled individuals and (2) estimation of sample haplotype frequencies, respectively. Most of the human haplotype blocks or genes are larger than 5 kb and thus the haplotyping methods must be capable of observing large genomic distances. They also should be possible to carry out in an accurate, cost-effective, and high-throughput manner to allow large-scale haplotyping. Moreover, they should permit the direct observation of individual haplotypes as this is important to understanding of an individual's risk of any given disease as well as drug side effects.

Both molecular and computational methods are available for haplotyping, each of which has its own advantages and disadvantages. In principle, molecular haplotyping represents a better approach since it can be performed on individual patients. However, existing molecular techniques all have limitations when applied to large-scale haplotyping. In general, they can be classified into two groups. The first group includes heteroduplex analysis [5], mismatch detection [6], and PCT bases allele discrimination techniques [7-10], all of which suffer from the fact that they can only determine the haplotype of a few kb distances in a chromosome, much smaller than many haplotype blocks and genes. In principle, some of these methods including long-range PCR based intramolecular ligation [11] can haplotype longer distances of DNA but at the expense of becoming very labor intensive. The second group of methods including cloning and physical separation of chromosome are limited by the fact that they are not easy to carry out in an automated and high-throughput manner [12-17] though they can reveal long distance haplotypes. Several new technologies have been proposed [18-20]. For example, methods such as rolling circle amplification [18] and nanotube atomic force microscopy [19] can haplotype sequence large blocks, but they have yet to be utilized in large-scale haplotyping. Boehnke et al. presented a method for the isolation of entire chromosomes, however that procedure does not seem to be easy to implement to deal with thousands of individuals [20]. Clearly, existing molecular methods do not adequately meet the requirements of large-scale haplotyping except when the sequence block is only a few kb long.

Because of limitations of existing molecular methods, haplotype structure has been traditionally deduced by computational methods in which haplotyping is achieved by genotyping with an assistance of statistical estimation. The two most popular methods are the parsimony approach developed by Clark [21] and maximum likelihood implemented via the expectation maximization (EM) algorithm [22-25], respectively. Clark's algorithm begins by listing all haplotypes that must be present unambiguously in the sample. Once this list of known haplotypes has been constructed, the haplotypes on this list are considered to see whether any of the unresolved genotypes can be resolved into a known haplotype. The algorithm continues cycling until all genotypes are resolved. Several problems can arise with this procedure, including the possibility of never being able to start the iterative algorithm because of the absence of any unambiguous individuals [21]. EM is a way of attempting to find the set of population haplotype frequencies that maximizes the probability of observing the genotypes. The EM algorithms are often limited in the size of problems they can tackle. For example, they are impracticable for sequence data containing individuals whose phase is ambiguous at more than 30 sites. Similarly, they cannot cope with larger number of linked SNPs. Several improved algorithms have been developed [26-30]. For example, Stephens et al. present a method by exploiting ideas from population genetics and coalescent theory that make prediction about the patterns of haplotypes to be expected in natural populations [26]. A novel feature is that it estimates the uncertainty associated.

Compared with existing molecular methods, computational methods can actually reveal the haplotyping structure of long genomic distances of DNA. However, they suffer from the uncertainty of knowing an individual's full haplotypes due to their statistical nature. Computational methods do not work well when analyzing large number of heterozygous SNPs without an assistance of molecular haplotyping [31]. They also often require collecting and genotyping DNA from family members. In some applications, molecular haplotyping is needed to confirming at least the part of haplotypes reconstructed to assure the accuracy and robustness of computational haplotyping [26, 31, 32].

Clearly, there is an urgent need in the art for haplotyping nucleic acids, particularly genomic DNA, in an accurate, low-cost, and high-throughput manner.

### SUMMARY OF THE INVENTION

The present invention provides methods for determining the haplotype structure of a nucleic acid target site comprising two or more single nucleotide polymorphisms (SNPs) which comprise different alleles or nucleotides, referred to hereinafter as the “SNPs of interest”. The method is particularly useful for determining the haplotype of target sites in which the SNPs of interest are separated by 100 kilobases or more. The method comprises preferentially extracting one allelic variant of a nucleic acid comprising the target site from a nucleic acid sample obtained from a subject to provide an enriched nucleic acid fraction in which the amount of one of the allelic variants of the nucleic acid, referred to hereinafter as the “enriched allelic variant” is from 1.5 to 100 times greater, preferably from 3 to 10 times greater, more preferably from 3 to 6 times greater, than the amount of the other allelic variant of the nucleic acid, referred to hereinafter as the “comparison allelic variant”, polymerase chain reaction (PCR) amplifying two or more of the SNPs of interest in the enriched nucleic acid fraction to provide a sample of PCR amplification products in which the amount, level or concentration of the PCR amplification products derived from the enriched allelic variant is greater than the amount, level or concentration of the PCR amplification products derived from the comparison allelic variant, and analyzing the PCR amplification products to identify the nucleotides in each of said two or more SNPs of interest that are present at a higher level or at a lower level than the other nucleotides in each of said two or more SNPs of interest and thus are located on the same allelic variant, i.e., the enriched allelic variant or the comparison allelic variant, respectively. Such analysis can be conducted using any genotyping procedure that allows one to determine the relative abundance of each nucleotide in each SNP of interest that is present in the PCR amplification products.

In another aspect, the method also comprises a step of determining the genotype of the allelic variants comprising the target site before extracting one of the allelic variants from the original nucleic acid sample.

The enriched allelic variant is preferentially extracted from the original nucleic acid sample using an allele-specific hybridization probe that is fully complementary to the sequence spanning one of the alleles of a SNP site that is located within or close to the target site in the allelic variants and that comprises two different alleles, referred to hereinafter as the hybridization SNP site. Hybridization is carried out under the conditions that allow the allele-specific hybridization probe to preferentially hybridize to one of the alleles of the hybridization SNP site as opposed to the other allele of the hybridization SNP site. In certain preferred embodiments, the hybridization probe also comprises a first binding molecule that binds the allele-specific probe to a solid substrate or to a second binding molecule for binding the hybridization probe and any nucleic acid that is bound thereto to a solid substrate. Thus, as shown in FIG. 1, a solid-phase extraction procedure can be used to preferentially extract one of the allelic variants from the original nucleic acid sample. The nucleic acid fraction that is extracted from the original nucleic acid sample comprises a greater amount of the enriched allelic variant relative to the comparison allelic variant, and thus, as shown in FIG. 1, the enriched allelic variant is preferentially used as the template in the PCR amplification step of the present method.

Additionally provided are kits for determining the haplotype structure of particular target sites or regions within a nucleic acid. The kits comprise a first allele specific hybridization probe that is completely complementary to a sequence spanning one of the alleles of a SNP located within or close to the target site of the targeted nucleic acid and a second allele-specific hybridization probe that is completely complementary to a sequence spanning the other allele of such SNP. The first hybridization probe also comprises a first binding molecule. The kit also comprises a solid support having attached thereto a second binding molecule which specifically binds to the first binding molecule and one or more primer set for PCR amplifying two or more SNPs within the target site of the nucleic acid.

### BRIEF DESCRIPTION OF THE FIGURES

FIG. 1 is a schematic illustration of one embodiment of the present method. Such method involves hybridization of an allele-specific probe comprising a binding molecule that allows for preferential extraction of one of the allelic variants of a nucleic acid comprising two SNPs of interest. As shown in the figure, such extraction can be a solid phase extraction. Thereafter, the nucleic acid fraction comprising the enriched allelic variant and the non-enriched allelic variant is genotyped. The haplotype structure of the enriched allelic variant and the non-enriched allelic variant is deduced based on the fact that after enrichment, the detection signal of any nucleotide in the sequence of the enriched allelic variant will be stronger than that of the corresponding nucleotide in the non-enriched allelic variant.

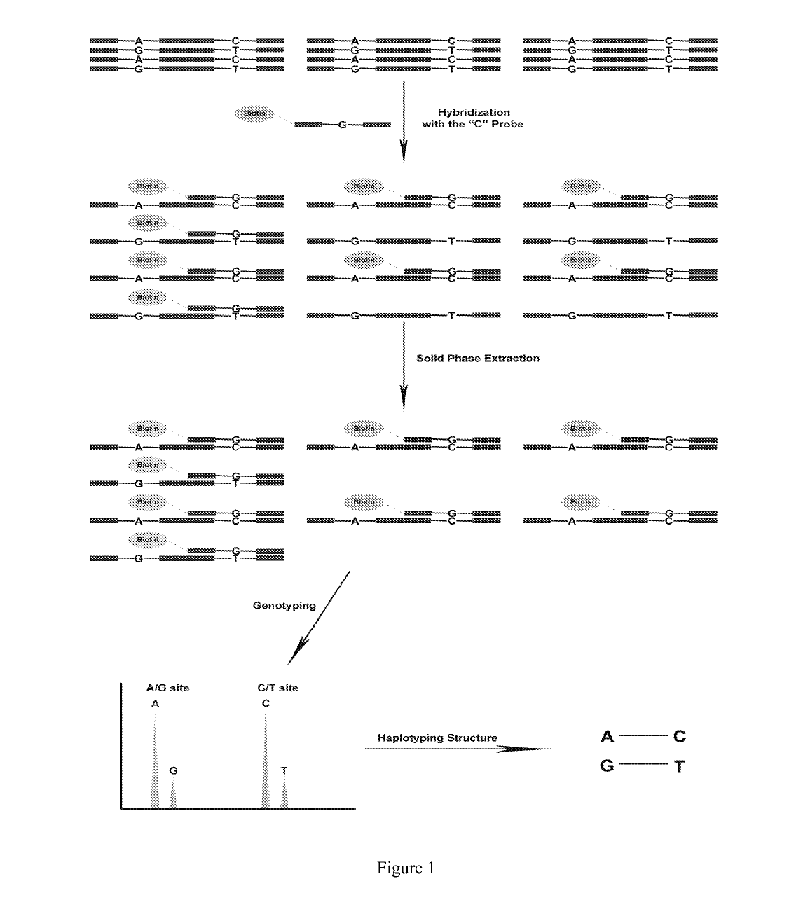

FIG. 2. is a graph showing the intensity levels of the alleles at SNP site rs1160985 in a DNA sample from a single individual: (a) before enrichment; and (b) after enrichment. Note that the signal intensity of the T allele of this SNP site has become much stronger in Spectrum (b) than in Spectrum (a), indicating successful enrichment of the T allele.

FIG. 3. is a graph showing the intensity levels of the alleles of SNP site rs1305062 in DNA samples obtained from two individuals after enrichment of the T allele of rs1160985. Note that the signal of the C nucleotide of rs1305062 becomes stronger in Spectrum (a), revealing a haplotype structure of T-C/C-G, while the signal of the G nucleotide dominates in Spectrum (b), indicating a T-G/C-C haplotype structure.

FIG. 4. is a graph showing the intensity levels of the alleles of SNP sites rs370705 and rs5167 in a single individual after enrichment of the T allele of rs1160986 using a PNA probe. Note that the signals of both the T nucleotide of rs370705 (Spectrum a) and the G nucleotide of rs5167 (Spectrum b) became stronger, indicating that this individual has a haplotype structure of T-T-G/C-C-T at SNPs of rs370705, rs1160985, and rs5167.

FIG. 5. is a photograph of an agarose gel showing the result of PCR amplification of a fragment containing rs1060985 after enrichment of the T allele of rs1060985. Note that all ten extractions were performed under the identical conditions and yielded the same haplotypes.

### DETAILED DESCRIPTION OF THE INVENTION

### Definitions

The term “a” or “an” as used herein means one or more. As used herein “another” means at least a second or more.

The term “allele” as used herein refers to one of a pair of autosomal chromosomes (or fragments thereof) that are present in organisms that sexually reproduce. Thus, the term allele as used herein can refer to one of two genes, or to one of two nucleotides that occupy the same position (locus) on a chromosome. The two alleles at each locus in the chromosome or chromosomal fragment may be the same or different. If the alleles at the same locus are the same, the individual or cell is referred to as homozygous for this allele. If the alleles at the same locus are different, the individual or cell is referred to as heterozygous for this allele.

The term “allelic variant” as used herein refers to a chromosome or chromosomal fragment in which the nucleotide sequence of one of the two copies or alleles of the chromosome or chromosomal fragment is different from the nucleotide sequence of the other copy or allele of the chromosome or chromosomal fragment.

The term “haplotyping” as used herein refers to a process of determining which alleles of two or more SNPs are located on the same chromosome. Each chromosome will have its own haplotype for the two SNP loci, therefore, each individual is expected to possess two haplotypes. The term haplotype is derived from the phrase “haploid genotype” and refers to the allelic constitution of a single chromosome or chromosomal region at two or more loci.

As used herein, “hybridization” refers to the formation of a complex structure, typically a duplex structure, by nucleic acid strands, e.g. single strands, due to complementary base pairing. Hybridization can occur between exactly complementary nucleic acid strands or between nucleic acid strands that contain minor regions of mismatch. Hybridization conditions should be sufficiently stringent that there is a difference in hybridization intensity between alleles. Hybridization conditions, under which a probe will preferentially hybridize to the exactly complementary target sequence are well known in the art (Sambrook et al., Molecular Cloning—A Laboratory Manual, Third Edition, Cold Spring Harbor Press, N.Y., 2001). Stringent conditions are sequence dependent and will be different in different circumstances. Generally, stringent conditions are selected to be about 5° C. lower than the thermal melting point (Tm) for the specific sequence at a defined ionic strength and pH.

The present invention provides methods of haplotyping nucleic acids that comprise two or more SNPs of interest. The methods of the invention are useful for obtaining haplotype information for any type of DNA-containing organism, including bacteria, virus, fungi, animals, including vertebrates and invertebrates, and plants. All references cited herein are specifically incorporated herein by reference.

The methods of the invention involve analysis of at least two SNPs of interest to identify the haplotype. The two SNPs may be referred to herein as the first SNP and the second SNP. The reference to a first or second SNP does not provide an indication of the order of the SNPs on the nucleic acid. The methods of the present invention are particularly useful for haplotyping nucleic acids in with the SNPs of interest are separated by a large number of kilobases, for example, 100 or more kilobases.

In one aspect, the method of the present invention comprises a step of extracting one allelic variant of a nucleic acid from an original nucleic acid sample comprising two allelic variants of the nucleic acid to provide an enriched nucleic acid fraction in which the amount of one of the allelic variants of the nucleic acid is 1.5 to 100, preferably from 3 to 10, more preferably 3 to 6 times, greater than the amount of the other allelic variant in the enriched nucleic acid fraction.

In a first embodiment, the enriched nucleic acid fraction contains the nucleic acid molecules that have been extracted from the original nucleic acid sample. The enriched nucleic acid fraction, preferably, is then PCR amplified to provide a PCR product sample in which the amount, level, or concentration of the PCR products that are derived from the extracted allelic variant is greater, preferably from about 1.5 to 100 times greater, more preferably from 3 to 10, most preferably from 3 to 6 times greater, than the amount, level or concentration of the PCR products that are derived from the allelic variant that has not been extracted from the original nucleic acid samples. The PCR products are than analyzed to identify the nucleotides in each of the two or more SNPs of interest that are present at a higher level or at a lower level than the other nucleotides in each of said two or more SNPs of interest, and thus are located on the same allelic variant, i.e., the extracted allelic variant or the non-extracted allelic variant, respectively.

In a second embodiment, the enriched nucleic acid fraction contains the nucleic acid molecules that have not been extracted from the original nucleic acid sample. The enriched nucleic acid fraction, preferably, is then PCR amplified to provide a PCR product sample in which the amount, level, or concentration of the PCR products that are derived from the non-extracted allelic variant is greater, preferably from about 1.5 to 100 times greater, more preferably from 3 to 10 times greater, most preferably from 3 to 6 times greater, than the amount, level or concentration of the PCR products that are derived from the allelic variant that has been extracted from the original nucleic acid samples. The PCR products are than analyzed to identify the nucleotides in each of the two or more SNPs of interest that are present at a higher level or at a lower level than the other nucleotides in each of said two or more SNPs of interest, and thus are located on the same allelic variant, i.e., the non-extracted allelic variant or the extracted allelic variant, respectively.

Extraction of the Nucleic Acid Variant from a Nucleic Acid Sample

In one aspect, the initial step of the present method involves extracting an allelic variant of a nucleic acid that comprises two or more SNPs of interest from a nucleic acid sample obtained from a DNA-containing subject, particularly a human subject. The nucleic acid sample can be obtained from any suitable source, such as for example, blood, eye fluid, cerebral spinal fluid, milk, ascites fluid, synovial fluid, peritoneal fluid, amniotic fluid, tissue, cell cultures, products of an amplification reaction and the like, environmental sources, and forensic sources including sewage and biological material deposited in or on cloth. In another aspect, the initial step of the present method comprises genotyping the nucleic acid of the subject to identify SNPs within and, optionally, near the target site that comprise two different alleles.

The original nucleic acid sample can contain intact nucleic acids (i.e., as they exist in the subject's cells), or can contain fragments of the nucleic acids. In this regard, fragmented nucleic acids are preferably relatively large so that it is less likely that a break or shear will occur between the SNPs of interest, which can destroy the haplotypic information encoded or contained within the target site. Therefore, the nucleic acids of the sample preferably are not so degraded that the distance between the first and second SNPs is greater than the median length of nucleic acid fragments in the sample. Similarly, the sample is preferably processed, if at all, so as to avoid excessive and unsuitable shearing or breakage of the nucleic acids in the sample. In contrast, however, some nucleic acid shearing can be advantageous because of its effect on the fluid dynamics of the sample containing the nucleic acid. In any event, it is difficult to prevent entirely the shearing of large nucleic acids, and it is not necessary to entirely prevent such shearing. Suitable methods for obtaining nucleic acids directly or indirectly from organisms that produce nucleic acid fragments of suitable sizes are well known in the art.

Other nucleic acids from the subject also can be used. For example, when two or more SNPs of interest are present in mRNA of the subject, the mRNA can be used in the present method. Because mRNA is unstable, it is preferably to reverse transcribe the mRNA to cDNA prior to extraction of one of the allelic variants from the sample. The original nucleic acid sample also can comprise cDNA, the preparation of which is frequently an initial step in the amplification of mRNA.

In a preferred embodiment, the original nucleic acid sample is genomic DNA. Genomic DNA comprises the entire genetic component of a species excluding, applicable, mitochondrial and chloroplast DNA. Of course, the methods of the invention can also be used to analyze mitochondrial, chloroplast, etc., DNA as well.

In one embodiment, one of the allelic variants is extracted from the original nucleic acid sample using an allele-specific probe, i.e., a probe that is fully complementary to sequence spanning one of the alleles of a heterozygous SNP site that is located within or near the target site of the allelic variant. The allele-specific probe can be an oligonucleotide, a modified oligonucleotide, or an analog of an oligonucleotide, such as a peptide nucleic acid (PNA) or a locked nucleic acid (LNA), which can preferentially hybridize with only one of the two alleles of the hybridization SNP site. For clinical applications, a standard panel of several SNP sites with a minor allele frequency close to 0.5 can be selected within or near the target site.

To enhance preferential hybridization of the oligonucleotide probe to the targeted SNP hybridization site, it is preferred that the Tm of the oligonucleotide probe be less than 60 degree, which can be achieved by shortening the probe or altering its sequence. It is also preferred that the SNP sites selected for hybridization provide a large change in Tₘ with one-base mismatch. In general, the stability of the hybrid follows the order of G-C>A-T>G-G>G-T=G-A>T-T=A-A>T-C>A-C>C-C, which provides guidance for designing the oligonucleotide probe. In certain embodiments of the present method, a competitor oligonucleotide or analog which comprises a sequence that is complementary to a sequence encompassing the other allele of the targeted SNP hybridization site is included in the hybridization step to enhance the preferential extraction of one of the allelic variants from the original nucleic acid sample. Methods for making the allele-specific probes and the competitor oligonucleotide or analog are known in the art.

The allele-specific probe may be attached directly or indirectly to the surface of a solid substrate and used for solid phase extraction of one of the allelic variants containing the target site from the original nucleic acid sample. Alternatively and preferably, the allele specific probe may be attached to a first binding molecule which is capable of binding to a second binding molecule that is directly or indirectly attached to a solid substrate. Examples of first and second binding molecules include, but are not limited to, biotin and avidin, antigens, such as fluorescein, and antibodies, such as anti-fluorescein antibodies, and nucleic acids that can specifically hybridize with nucleic acids attached to a surface. A surface, as used herein, refers to any type of solid support material to which a molecular component such as the probe or second binding molecule is capable of being fixed. Surfaces include, for instance, single or multi-well dishes, chips, slides, membranes, beads, agarose or other types of solid support mediums.

In one embodiment of the present invention, the allele-specific probe and, optionally, the competitor oligonucleotide or analog are reacted with the original nucleic acid sample under hybridization conditions that allow the allele-specific probe to preferentially hybridize to one of the allelic variants of the nucleic acid comprising a target site, and the competitor oligonucleotide or analog, if present, to preferentially hybridize with the other allelic variation of the nucleic acid molecule comprising the target site to provide an enriched nucleic acid fraction in which the concentration, level or amount of one of the allelic variants of the nucleic acid comprising the target site is 3, 4, 5, 6, 7, 8, 9, or 10, preferably from 3-6 times greater than the other allelic variant. In certain embodiments, the enriched nucleic acid fraction is bound to the solid substrate and the non-enriched nucleic acid fraction is the nucleic acid fraction that is not bound to the substrate. As shown in the example 5 below, good results have been obtained employing a biotinylated allele-specific oligonucleotide and a competitor oligonucleotide.

In other embodiments, the enriched nucleic acid fraction is in the nucleic acid fraction that is not bound to the solid substrate and the non-enriched nucleic acid fraction is the fraction that is bound to the substrate.

Amplification Methods.

It may be desirable to amplify the nucleic acids in the enriched nucleic acid fraction before determining the haplotypes of the enriched allelic variant and non-enriched nucleic variant. In the present case nucleic acid amplification proportionately increases the number of copies of the products derived from the enriched allelic variant and the non-enriched allelic variant. Any amplification technique known to those of skill in the art may be used in conjunction with the present invention including, but not limited to, polymerase chain reaction (PCR) techniques. PCR may be carried out using materials and methods known to those of skill in the art.

PCR amplification generally involves the use of one strand of a nucleic acid sequence as a template for producing a large number of complements to that sequence. The template may be hybridized to a primer having a sequence complementary to a portion of the template sequence and contacted with a suitable reaction mixture including dNTPs and a polymerase enzyme. The primer is elongated by the polymerase enzyme producing a nucleic acid complementary to the original template.

For the amplification of both strands of a double stranded nucleic acid molecule, two primers may be used, each of which may have a sequence which is complementary to a portion of one of the nucleic acid strands. The strands of the nucleic acid molecules are denatured—for example, by heating and the process is repeated, this time with the newly synthesized strands of the preceding step serving as templates in the subsequent steps. A PCR amplification protocol may involve a few to many cycles of denaturation, hybridization and elongation reactions to produce sufficient amounts of the desired nucleic acid.

Template-dependent extension of primers in PCR is catalyzed by a polymerase enzyme in the presence of at least 4 deoxyribonucleotide triphosphates (typically selected from dATP, dGTP, dCTP, dUTP and dTTP) in a reaction medium which comprises the appropriate salts, metal cations, and pH buffering system. Suitable polymerase enzymes are known to those of skill in the art and may be cloned or isolated from natural sources and may be native or mutated forms of the enzymes.

The nucleic acids used in the methods of the invention may be labeled to facilitate detection in subsequent steps. Labeling may be carried out during an amplification reaction by incorporating one or more labeled nucleotide triphosphates and/or one or more labeled primers into the amplified sequence. The nucleic acids may be labeled following amplification, for example, by covalent attachment of one or more detectable groups. Any detectable group known to those skilled in the art may be used, for example, fluorescent groups, ligands and/or radioactive groups.

In a preferred embodiment of the present method, the enriched nucleic acid fraction subjected to PCR amplification to proportionately increase the levels of the SNP alleles in the enriched allele variant and the SNP alleles in the non-enriched allelic variant for subsequent genotyping. Depending on the distance between the SNPs in the target site, e.g. SNP1 and SNP2, one or more primer sets are used to PCR amplify the enriched nucleic acid fraction. For example, if the SNPs of interest are within one kilobase of each other a primer set comprising a first primer and a second primer that flank all of the SNPs of interest is used. Such procedure results in a single PCR product comprising one of the alleles for each of the SNPs of interest within the enriched allelic variant and a single PCR product comprising the other alleles for each of the SNPs of interest within the non-enriched allelic variant. Since, the enriched nucleic acid fraction comprises from 1.5 to 100 times, preferably 3 to 10 times, more preferably from 3 to 6 times more, of the enriched allelic variant than the non-enriched allelic variant the PCR amplification results in the production of proportionately more of the PCR product or products derived from the enriched allelic variant. Alternatively, if the SNPs of interest are more than one kilobase apart, it is preferable to use multiple primer sets in which the first primer set flanks the first SNP of interest, the second primer set flanks the second SNP of interest, the third primer set flanks the third SNP of interest, etc. In the latter case, multiple PCR products are produced, and each PCR product comprises one or a few SNPs of interest. Again, the PCR products that are derived from the enriched allelic variant are present in greater abundance than the PCR products that are derived from the non-enriched allelic variant.

Analysis of the PCR Products

The PCR products are then genotyped by any genotyping method to identify the nucleotides of each SNP that are present are higher levels and thus, are located on the enriched allelic variant, as well as the nucleotides of each SNP that are present at lower levels, and thus, are located on the non-enriched allelic variant. The alleles that are located on the enriched allelic variant form one of the haplotypes of the targeted nucleic acid, and the alleles that are located on the non-enriched allelic variant form the other haplotype of the targeted nucleic acid.

Suitable methods for genotyping the PCR products include, but are not limited to, hybridization, primer extension, MALDI-TOF, HPLC, solution phase detection, Taqman™, and fluorescence detection.

Primer extension is performed by hybridizing primers which flank but do not span the second SNP, performing a primer extension reaction to produce a PCR product. The primers may hybridize directly to the nucleic acid adjacent to the SNP site or they may hybridize to a site which is some distance away. It is possible to determine which allele is present in the nucleic acid sample in one of several ways. For instance, if one possible allele is a G at the SNP site then a labeled G can be added to the primer extension mixture instead of an unlabeled G. In some cases the labeled nucleotide is a dideoxynucleotide which will stop the production of the strand being created. The label may be any type of detectable label, e.g., a fluorescent label or a binding partner, e.g., biotin.

MALDI-TOF (matrix-assisted laser desorption ionization time of flight) mass spectrometry provides for the spectrometric determination of the mass of poorly ionizing or easily-fragmented analytes of low volatility by embedding them in a matrix of light-absorbing material and measuring the weight of the molecule as it is ionized and caused to fly by volatilization. Combinations of electric and magnetic fields are applied on the sample to cause the ionized material to move depending on the individual mass and charge of the molecule. U.S. Pat. No. 6,043,031, issued to Koster et al., describes an exemplary method for identifying single-base mutations within DNA using MALDI-TOF and other methods of mass spectrometry. Other methods are described in U.S. Pat. Nos. 6,002,127; 5,965,363; 5,905,259; 5,885,775; and 5,288,644, each of which is incorporated by reference. One preferred method is the MALDI-TOF VSET method which is described in U.S. Pat. No. 6,479,242, and is specifically incorporated herein in its entirety.

HPLC (high performance liquid chromatography) is used for the analytical separation of bio-polymers, based on properties of the bio-polymers. HPLC can be used to separate nucleic acid sequences based on size and/or charge. A nucleic acid sequence having one base pair difference from another nucleic acid can be separated using HPLC. Thus, nucleic acid samples, which are identical except for a single allele may be differentially separated using HPLC, to identify the presence or absence of a particular allele. Preferably the HPLC is dHPLC (denatured HPLC). dHPLC involves the denaturation of the nucleic acid sample, followed be a reannealing step where the nucleic acid can assume a secondary structure, which will differ somewhat in nucleic acid samples having different alleles.

The invention involves improved methods for screening DNA to identify polymorphic haplotypes and to enable identification of haplotypes associated with predisposition to diseases as well as other genetically associated traits In general, the present haplotyping methods are useful in linkage disequilibrium studies for the analysis of complex traits to localized genes involved in diseases such as diabetes, multiple sclerosis, and asthma; diagnostic analysis to determine the presence or absence of a predisposing disease haplotype or other trait; pharmacogenomic analysis to identify haplotypes that correlate with either positive or negative responses to drugs and development; genome-wide scan studies for complex trait analysis using SNP haplotypes, instead of single SNPs, to increase the statistical power; etc.

The haplotyping methods of the invention are useful for identifying both normal phenotypes and disease phenotypes. Thus, the methods for the invention are useful for identifying traits such as eye color as well as for diagnostics to determine presence or absence of predisposing disease haplotype in a subject. Some diseases which are known to have a genetic element include colon cancer, breast cancer, cystic fibrosis, neurofibromatosis type 2, LiFraumeni disease, VonHippel-Lindau disease, thalassemia, ornithine, transcarbamylase deficiency, hypoxanthine-guanine-phosphoribosyl-transferase deficiency, phenylketonuria, etc.

Identification of haplotypes associated with phenotypic traits is useful for many purposes in addition to identifying predisposition to disease. For example, identification of a correlation between susceptibility to a particular drug or a therapeutic treatment and specific genetic alterations is particularly useful for tailoring therapeutic treatments to a specific individual. The methods are also useful in prenatal screening to identify whether a fetus is afflicted with or is predisposed to develop a serious disease. Additionally, this type of information is useful for screening animals or plants bred for the purposes of enhancing or exhibiting desired characteristics.

### EXAMPLES

The following examples contained herein are intended to illustrate but not limit the invention.

Methods and Materials:

Human genomic DNA was extracted from blood samples using GenomicPrep Blood DNA Isolation kit (Amersham Pharmacia Biotech, Piscataway, N.J.). To extract one allelic variant of the nucleic acid comprising the target site, 5 ng of genomic DNA, 0.1 pmole of the biotinylated PNA probe, or 5 pmole of biotinylated oligo probe and 25 pmole of the unbiotinylated oligo probe, and 5 μL of high salt buffer (0.1M Na₂EDTA, 0.2M sodium phosphate, 0.25% SDS, pH 8.0) were mixed together in 25 μL. The mixture was first denatured at 95° C. for 10 min, followed by reducing the temperature at a rate of 0.1° C./sec to the hybridization temperature (52° C.), the hybridization temperature was, then, maintained at that temperature for 30 minutes. Thereafter, the mixture along with 5 μL of magnetic beads (Dynal Biotech, Lake Success, N.Y.) was added to 25 μl of B&W buffer and incubated at room temperature for 30 minutes. The supernatants were removed, followed by washing the beads. The purified beads containing enriched DNA were resuspended in 10 μL of water, followed by heating it at 95° C. for 10 minutes to remove the enriched DNA fraction from the beads.

1 μL (10%) of the enriched DNA fraction was added to a PCR tube. PCR was performed in 25 μL using 5 pmole of each forward and reverse primers, 0.2 mM of each dNTPs, 2 mM of MgCl₂, 1× AmpliTaq Gold PCR buffer and 1 unit of AmpliTaq Gold DNA polymerase (PE Biosystems). After denaturation of 95° C. for 10 min (it is noted that at this temperature, the DNA templates will be separated from the beads), PCR was performed for 42 cycles consisting of 30 seconds at 95° C., PCR primer annealing temperature for 30 sec, and 60 sec at 72° C. with a final extension of 5 min at 72° C.

PCR products were treated with Alkaline Phosphatase and Exonuclease I (Amersham Pharmacia Biotech) for 60 min at 37° C. followed by 15 min at 80° C. prior to primer extension. 5 μL of the treated PCR products were used as the templates of VSET-based primer extension, which was performed in 10 μL using 0.1 mM of the required dNTPs, 0.025 mM of the required ddNTPs, 2.5 pmole of extension primers, 0.5× ThermoSequenase buffer, and 0.5 unit of ThermoSequenase (Amersham Pharmacia Biotech). After an initial denaturation of 120 sec, extension was performed for 60 cycles of 92° C. (20 sec), primer annealing temperature (20 sec), and 72° C. (20 sec). The extension products were purified by ZipTip (Millipore, Bedford, Mass.) prior to MALDI-TOF analysis. The MALDI sample was prepared by mixing the purified extension products and matrix (saturated 3-hydropicolinic acid in a 1:1:2 mixture of water, CH₃CN, and 0.1M ammonium citrate). The sample was dried and then analyzed using MALDI-TOF.

### Example 1

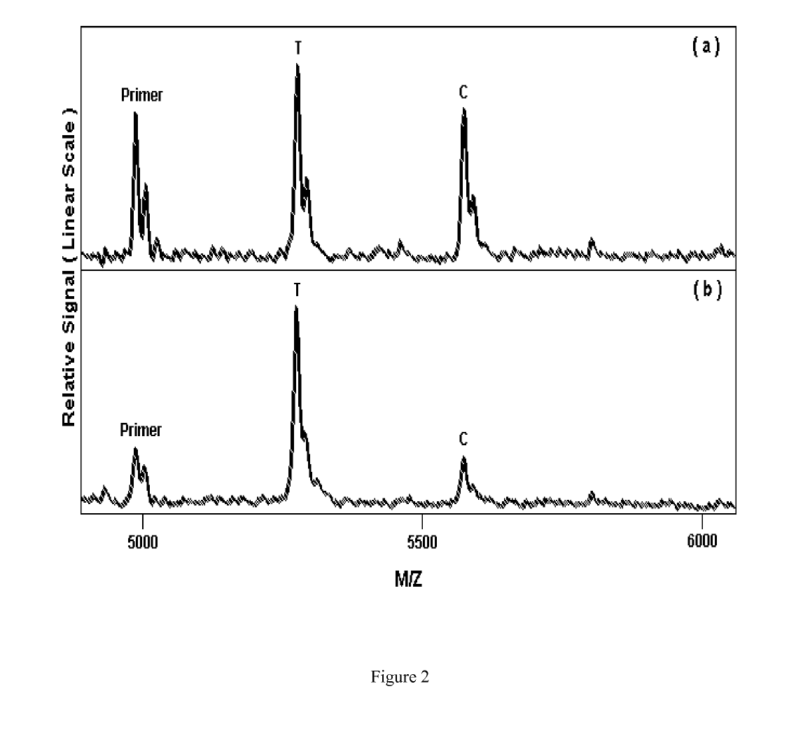

A PNA probe was used to preferentially extract an allelic variant comprising a block around ApoE4 [40,41]. A sequence spanning SNP rs1160985 (C/T) was selected as the targeted hybridization site and a PNA probe was used to hybridize with the T allele of this SNP. 12 individuals who are heterozygous at this site were examined. FIG. 2 shows the results of genotyping of an individual at this site before and after enrichment, respectively. Genotyping was carried out using the MALDI-TOF-based VSET assay as described in [38]. As shown in FIG. 2, the signal corresponding to the T allele became stronger after enrichment, suggesting successful enrichment of the sequences containing the T allele. These results indicate that any sequences containing the T allele of this SNP become more abundant than the corresponding sequences containing the C allele. The correct enrichment was achieved with all 12 individuals. In general, an enrichment yielding a 3:1 molar ratio of the enriched to the non-enriched alleles should be useful for this application. In fact, the presence of the signals arising from the non-enriched allele is very useful, as it can reveal the haplotype of another allele without additional genotyping.

### Example 2

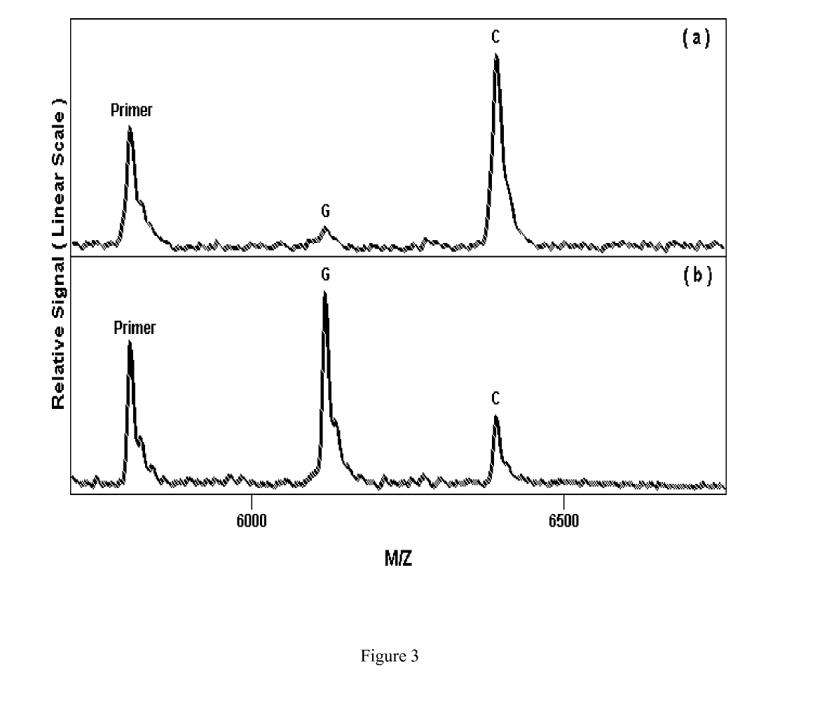

SNP rs1160985 and SNP rs1305062 (C/G), which is 2.1 kb away (3′ direction) from rs116098 were analyzed as described above using a PNA molecule as the allele-specific probe. The sequence containing the T allele of rs1160985 was enriched and thus a nucleotide in a sequence containing the T allele of rs1160985 should yield a stronger signal than does the corresponding nucleotide in a sequence containing the C allele. PCR was performed to amplify ˜200 bp fragment containing the locus of rs1305062 using the enriched nucleic acid fraction as template, followed by genotyping rs1305062. FIG. 3 shows the results of genotyping two individuals who have different haplotypes. The peak corresponding to the C allele of rs1305062 became stronger after enrichment in FIG. 3a, suggesting that the C allele of this individual is on the same chromosome containing the T allele of rs1160985. In other words, the haplotype of this individual is T-C/C-G at rs1160985 and rs1305062. FIG. 3b shows the result of haplotyping another individual, in which the peak of the G allele was stronger, revealing that the haplotype of this second individual is T-G/C-C, different from the haplotype of the first individual.

### Example 3

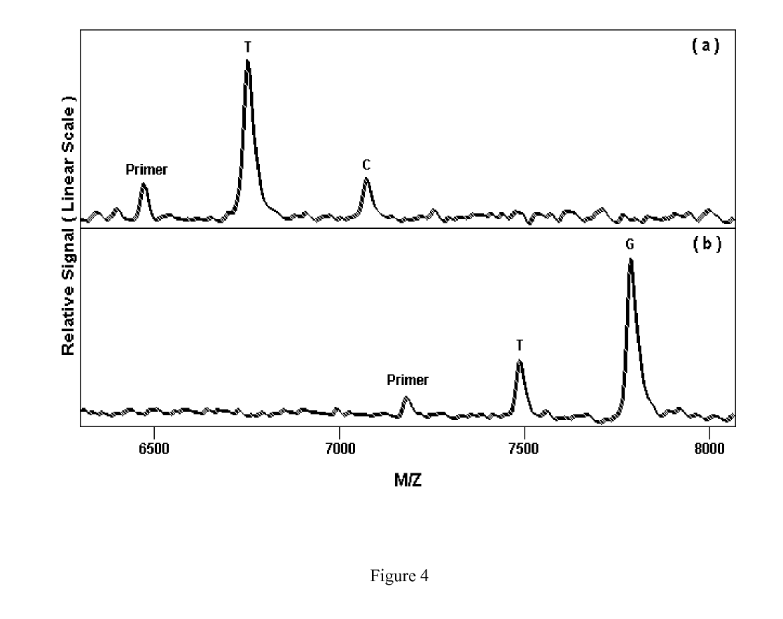

SNP rs1160985, SNP rs370705 (C/T, 22 kb upstream from rs1160985), and SNP rs5167 (T/G, 45 kb downstream from rs1160985) were analyzed using allele-specific PNA probes as described above. The allelic variant containing the T allele of rs1160985 was enriched. Once again, any nucleotide in the sequence containing the T allele of rs1160985 is expected to yield a more intense signal than that of the corresponding nucleotide in the C allele of rs160985. Only individuals who are heterozygous at all these three sites were analyzed. The genotyping of an individual after enrichment is displayed in FIG. 4. After enrichment, the signal of the T nucleotide of rs370705 became more intense in FIG. 4a, while the G nucleotide of rs5167 dominated in FIG. 4b, suggesting these nucleotides are present on the same chromosome containing the T allele of rs 1160985. In other words, this individual has a haplotype of T-T-G/C-C-T at SNPs of rs1160985, rs370705, and rs5167.

### Example 4

SNP rs1160985 was analyzed using an allele-specific oligonucleotide probe and a competitor oligonucleotide. Each extraction employed an allele-specific oligonucleotide probe that was complementary to one of the alleles of the heterozygous SNP site and a competitor oligonucleotide that was complementary to the other allele of the heterozygous SNP site. However, only the allele-specific probe was biotinylated, thus enriching only one allele. Since the Tₘ change of oligonucleotides sometimes may not be sufficiently large to discriminate two SNP alleles differing by one base [42], the allele-specific oligonucleotide probe and the competitor oligonucleotide were both used in the extraction step to enhance enrichment. The results indicated that the haplotype structures deduced by enriching the T allele of rs1160985 using an oligonucleotide probe and a competitor oligonucleotide were in a total agreement with those observed via enrichment of the T allele of the same SNP site using a PNA probe, demonstrating that an oligonucleotide probe can be used in the present method.

### Example 5

SNP rs370705, SNP rs5167, and SNP rs5167 were analyzed as described above using an allele-specific oligonucleotide probe and a competitor oligonucleotide. The haplotypes of two individuals were investigated. Table 1 lists the haplotyping results using rs1160985, rs370705, and rs5167 as the extraction sites, respectively. The capital letter indicates the nucleotide yielding a stronger signal in a given locus, while the uncapped letter stands for the nucleotide yielding a weaker signal at the same site. The haplotype structure of each individual was deduced on the basis that the nucleotides yielding a stronger signal should arise from a same chromosome (enriched), while all nucleotides having a weaker signal came from the other chromosome (unenriched). As seen from Table 1, the identical haplotype structures were deduced for the same individual, using three different extraction sites. Table 1 also shows that person 1 and person 2 have different haplotypes at SNPs of rs1160985, rs370705, and rs5167. This result clearly displays the effectiveness of oligonucleotide probes and the robustness of the present method.

TABLE 1
Haplotyping Results of Two Individuals Using Three Different Extraction Sites

| | rs1160985 (T) | | rs5167 (T) | | rs370705 (T) | |
|---|---|---|---|---|---|---|
| | Genotyping | | Genotyping | | Genotyping | |
| Extraction Site (Allele) | Person 1 | Person 2 | Person 1 | Person 2 | Person 1 | Person 2 |
| rs1160985 | T/c | T/c | C/t | T/c | T/c | C/t |
| rs370705 | T/c | C/t | C/t | C/t | T/c | T/c |
| rs5167 | G/t | T/g | T/g | T/g | G/t | G/t |
| Deduced Haplotype | T-T-G | T-C-T | t-t-g | T-C-T | T-T-G | t-c-t |
| | c-c-t | c-t-g | C-C-T | c-t-g | c-c-t | C-T-G |

SNPs of rs370705 and rs5167 are separated by 67 kb of genomic sequence, and they can be phased by using either SNP as the extraction site or targeted SNP hybridization site, suggesting that this method can haplotype a sequence of at least 134 kb in length. This is because if SNPs A, B, and C (where B is between A and C, and separated from A and C by the same distance) are heterozygous and we know the phase of A and B, and the phase of B and C, then we know the phase of SNP A and C. This sequence is about three times longer than the largest sequence (45 kb) haplotyped by other molecular methods [4]. This haplotyping capability is sufficient to cover the entire sequence of most genes. It is noted that this length should not be the limit. In principle, one should be able to haplotype a sequence of any distances using the present method, as long as the sample is not sheared or degraded to the extend that DNA molecules in the sample are all too short to contain the sequence of interest.

As shown above, the present method can reveal individual haplotypes in a sequence of ˜134 kb in length, about three times longer than the largest sequence (˜45 kb) haplotyped by a molecular method, and thus can be used in in clinic applications. In addition, the enrichment step of the present method is extremely simple, low-cost, and can be easily automated. After enrichment, haplotyping is essentially achieved through genotyping, and thus the present method can take advantages of the accurate, fast, low-cost, and robust features of the most advanced genotyping methods.

Compared with existing molecular methods, the present method offers a better way to reveal DNA haplotype structures of both short and long genomic distances in a more accurate, cost-effective, and high-throughput manner. In addition, the present method does not require complex software to deduce haplotypes, since it directly yields haplotypes. Thus, the present method should be useful in many fields including the discovery of new genes, drug development, pharmacogenetics, and personalized medicine.

### Example 6

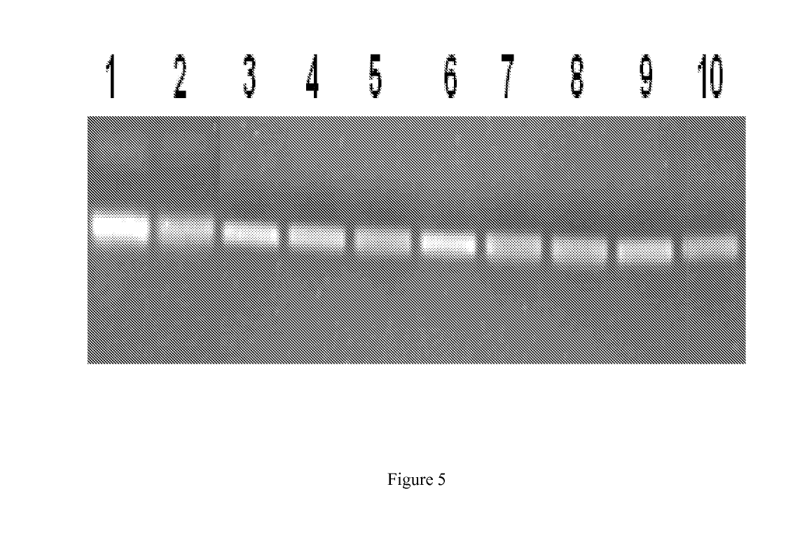

It is noted that the present method is extremely reproducible and easy to use. For example, we utilized the same extraction procedure on four different extraction sites, and were able to deduce the correct haplotype structure in each case. We also separately haplotyped a single individual ten (10) times using the same procedure and obtained the identical result. This demonstrates the robustness of this present method. Moreover, we have also studied the efficiency of allele-specific extraction and found that we could recover 30-50% of the targeted alleles. Therefore, we have been routinely using only 5 ng of genomic DNA samples for extraction and utilizing only 10% (equal to 0.5 ng of total genomic DNA) of the extraction DNA as the PCR template. For example, FIG. 5 shows the result of PCR amplification of a 200 bp fragment containing rs1060985 locus after the enrichment of the T allele of rs1060985. We repeated the extraction 10 times using the identical condition. In each extraction, a total of 5 ng of genomic DNA were extracted and 10% of the extracted DNA was subject to PCR. Lane 1 to 10 shows the PCR products of these ten (10) different extractions. Clearly, FIG. 5 shows that every extraction was successful, suggesting the effectiveness and robustness of the extraction procedure. We expect that 0.5 ng of genomic DNA should be sufficiently enough for allele-specific extraction, which is adequate for a general clinic application where at least several microliters of blood can be obtained.

### LITERATURE CITED

1. WWW.GENOME.GOV/PAGE.CFM?PAGEID=10005336.
2. D. B. Goldstein and M. E. Weale, Current Biology, 11, R576 (2001).
3. R. Judson, J. C. Stephens and A. Windemuth, Pharmacogenetics, 1: 15 (2000).
4. R. Judson and J. C. Stephens, Pharmacogenetics, 2: 7 (2001).
5. F. M. Chang and K. K. Kidd, Am J. Med. Genet., 74, 91 (1997).
6. U.S. Pat. No. 1,160,684 (2000).
7. G. Ruano and K. K. Kidd, Nucleic Acids Research, 17, 8392 (1989).
8. S. Michalatos-Beloin, S. A. Tishkoff, K. L. Bentley, K. K. Kidd and G. Ruano, Nucleic Acids Research, 24, 4841 (1996).
9. Y. Eitan and Y. Kashi, Nucleic Acids Research, 30, e62 (2002).
10. J. Tost, O. Brandt, F. Boussicault, D. Derbala, C. Caloustian, D. Lechner and I. G. Gut, Nucleic Acids Research, 30, e96 (2002).
11. O. G. McDonald, E. Y. Krynetski and W. E. Evans, Pharmacogenetics, 12, 93 (2002).
12. G. Ruano, K. K. Kidd and J. C. Stephens, Proc. Natl. Acad. Sci. USA, 87, 6296 (1990).
13. D. R. Cox, M. Rumeister, E. R. Price, S. Kim and R. M. Myers, Science, 250:245 (1990).
14. E. A. Steward, K. B. Mckusik, A. Aggarwal, E. Bajorek, S. Brady, A. Chu, N. Fang, D. Hadley, M. Harris et al., Genome Research, 7, 422 (1997).
15. M. S. Bradshaw, C. S. Shashikant, H. C. Belting, J. A. Bollekens and F. H. Ruddle, Proc. Natl. Acad. Sci. USA, 95, 4469 (1998).
16. N. Kouprina, L. Annab, J. Graves, C. Afshari, J. C. Barrett, M. A. Resnick and V. Larionov, Proc. Natl. Acad. Sci. USA, 95, 4469 (1998).
17. N. Papadopolous, F. S. Leach, K. W. Kinzler and B. Vogelstein, Nature Genetics, 11, 99 (1995).
18. P. M. Lizardi, X. Huang, Z. Zhu et al., Nature Genetics, 19, 225 (1998).
19. A. T. Woolley, C. Guillemette, L. Cheung, D. E. Housman and C. M. Lieber, Nature Biotechnology, 18, 760 (2000).
20. J. A. Douglas, M. Boehnke, E. Gillanders, J. M. Trent and S. B. Gruber, Nature Genetics, 28, 361 (2001).
21. A. G. Clark, Mol. Biol. Evol., 7, 111 (1990).
22. L. Excoffier and M. Slatkin, Mol. Biol. Evol., 12, 921 (1995).
23. M. Hawley and K. K. Kidd, J. Hered., 86, 409 (1995).
24. J. C. Long, R. C. Williams and M. Urbanek, Am. J. Hum. Genet., 56, 799 (1995).
25. D. Fallin and N. J. Schork, Am. J. Hum. Genet., 67, 947 (2000).
26. M. Stephens, N. J. Smith and P. Donnelly, Am. J. Hum. Genet., 68, 978 (2002).
27. J. R. O'Connell, Genetic Epidemiology, 19 (Suppl. 1), S64 (2000).
28. D. Qian and L. Beckmann, Am. J. Hum. Genet., 70, 1434 (2002).
29. K. Zhang, P. Calabrese, M. Nordborg and F. Sun, Am. H. Hum. Genet., 71:1386 (2002).
30. K. Rhode and R. Fuerst, Human Mutation, 17, 289 (2001).
31. A. G. Clark, K. M. Weiss, D. A. Nickerson et al., Am. J. Hum. Genet., 63, 595 (1998).
32. S. M. Fullerton, A. G. Clark, K. M. Weiss, D. A. Nickerson, S. L. Taylor, J. H. Stengard, V. Salomaa et al., Am. J. Hum. Genet., 67, 881 (2000).
33. C. Tong and L. M. Smith, Anal. Chem. 64, 2672 (1992).
34. X. Sun, H. Ding, K. Hung, and B. C. Guo, Nucleic Acids Research, 28, e68 (2000).
35. G. C. L. Johoson, L. Esposito, B. J. Barratt, A. N. Smith, J. Heward, D. D. Genova, H. Ueda, et al., Nature Genetics, 29, 233 (2001).
36. S. B. Gabriel, S. F. Schaffner, H. Nguyen, J. M. Moore, J. Roy, B. Blumenstiel, J. Higgins, et al., Science, 296, 2225 (2002).
37. D. E. Reich, M. Gargill, S. Bolk, J. Ireland, P. S. Sabeti, D. J. Richter, T. Layery, R. Kouyoumijan, S. F. Farhadian, R. Ward and W. S. Lander, Nature, 411, 199 (2001).
38. A. J. Jefferys, L. Kauppi and R. Neumann, Nature Genetics, 29, 217 (2001).
39. M. J. Daley, J. D. Rioux, S. F. Schaffner, T. J. Hudson and E. S. Lander, Nature Genetics, 29, 229 (2001),
40. J. D. Rioux, M. J. Daley, M. S. Silverberg, K. Lindblad, H. Steihart, X. Cohen, T. Delmonte, K. Kocher, et al., Nature Genetics, 29, 223 (2001).
41. P. C. Sabeti, D. E. Reich, J. M. Higgins, H. Z. Levine, D. J. Richter, S. F. Schaffner, S. B Gabrier, et al., Nature, 24, 419 (2002).
42. E. R. Martin, et al., Am. J. Hum. Genet., 67, 384 (2000).
43. WWW.NCBI.NLM.GOV/SNP.

What is claimed is:

1. A method of determining the haplotype structures of a nucleic acid comprising two or more single nucleotide polymorphisms (SNPs) of interest, wherein said SNPs of interest comprise different alleles, comprising:
obtaining an enriched nucleic acid fraction which comprises from 2 to 30 times more of one allelic variant of the nucleic acid than the other allelic variant of the nucleic acid; wherein said one allelic variant is an enriched allelic variant and the other allelic variant is a non-enriched allelic variant; and
genotyping the enriched nucleic acid fraction to identify the alleles in said two or more SNPs of interest that are present at a higher level or lower level than the other alleles in said two or more SNPs of interest, wherein the alleles that are present at a higher level are on the enriched allelic variant and form one haplotype and the alleles that are present at a lower level are on the non-enriched allelic variant and form the other haplotype.

2. The method of claim 1 wherein the enriched nucleic acid fraction is obtained by preferentially extracting one allelic variant of the nucleic acid from a nucleic acid sample obtained from a subject.

3. The method of claim 2 wherein said one allelic variant is preferentially extracted from the nucleic acid sample by contacting the nucleic acid sample with an allele-specific hybridization probe that is fully complementary to a sequence of one allele of a heterozygous SNP site that is located within or near the target site, wherein said allele specific hybridization probe is attached to a solid support or to a first binding molecule that is capable of binding to a second binding molecule that is attached to a solid support, and wherein the nucleic acid sample and the allele-specific hybridization probe are contacted under hybridization conditions that allow the allele-specific hybridization probe to preferentially hybridize with said one allele of said heterozygous SNP site.

4. The method of claim 3 wherein said allele-specific hybridization probe is attached to a first binding molecule.

5. The method of claim 4 wherein the first binding molecule is biotin or streptavidin and said second binding molecule is streptavidin or biotin, respectively.

6. The method of claim 2 wherein the enriched nucleic acid fraction comprises the nucleic acid molecules that are extracted from the nucleic acid sample and the level of the enriched allelic variant in the enriched nucleic acid fraction is from 1.5 to 100 times greater that the level of the non-enriched allelic variant in the enriched nucleic acid fraction.

7. The method of claim 2 wherein the enriched nucleic acid fraction comprises the nucleic acid molecules that are not extracted from the nucleic acid sample.

8. The method of claim 1 further comprising the step of amplifying the nucleic acids in the enriched nucleic acid fraction prior to identifying the alleles in said two or more SNPs of interest that are present at a higher level or lower level in the enriched nucleic acid fraction than the other alleles in said two or more SNPs of interest, wherein said amplification proportionately increases the amount of the enriched allelic variant and the non-enriched allelic variant in the enriched nucleic acid fraction.

9. The method of claim 2 further comprising the step of amplifying the nucleic acids in the enriched nucleic acid fraction prior to identifying the alleles in said two or more SNPs of interest that are present at a higher level or lower level in the enriched nucleic acid fraction than the other alleles in said two or more SNPs of interest, wherein said amplification proportionately increases the amount of the enriched allelic variant and the non-enriched allelic variant in the enriched nucleic acid fraction.

10. The method of claim 8 wherein the nucleic acids in the enriched nucleic acid fraction are amplified by a polymerase chain reaction (PCR) amplification procedure which employs one or more primer sets that hybridize to sequences flanking said two or more SNPs of interest.

11. The method of claim 9 wherein the nucleic acids in the enriched nucleic acid fraction are amplified by a polymerase chain reaction (PCR) amplification procedure which employs one or more primer sets that hybridize to sequences flanking said two or more SNPs of interest.

12. The method of claim 2 wherein the allele-specific hybridization probe is an oligonucleotide, a peptide nucleic acid or a locked nucleic acid.

13. The method of claim 1 wherein the nucleic acid sample is a genomic DNA sample.

14. The method of claim 2 wherein the nucleic acid sample is a genomic DNA sample.

15. The method of claim 2 wherein the allele-specific hybridization probe is an oligonucleotide that is attached to a first binding molecule and said nucleic acid sample is contacted with both the allele-specific hybridization probe and a competitor oligonucleotide that hybridizes to the other allele of the heterozygous SNP site and that is not attached to the first binding molecule.

16. The method of claim 1 wherein the enriched nucleic acid fraction comprises 3 to 6 times more of the enriched allelic variant than the non-enriched allelic variant.

17. The method of claim 2 wherein the genotype of the nucleic acid comprising the target site is determined before one allelic variants of the nucleic acid is extracted from the original nucleic acid sample.

18. A method of determining the haplotype structures of two allelic variants of a chromosome or chromosomal fragment comprising two or more single nucleotide polymorphisms (SNPs) of interest, wherein said SNPs of interest comprise different alleles, said method comprising:
preferentially extracting one of said two allelic variant from an original nucleic acid sample comprising said two allelic variants of said chromosome of chromosomal fragment to provide an enriched sample in which the level of the preferentially extracted allelic variant is from 2 to 30 times greater than the level of the allelic variant that is not preferentially extracted from the sample;
PCR amplifying the enriched sample to proportionately increase the level of the allelic variant that is preferentially extracted from the sample and the level of the allelic variant that is not preferentially extracted from the sample; and
identifying the alleles of the SNPs of interest that are present at higher levels in the amplified enriched sample and are located on the allelic variant that is preferentially extracted from the original nucleic acid sample; and
identifying the alleles of the SNPs of interest that are present at lower levels in the amplified enriched sample and are located on the allelic variant that is not preferentially extracted from the original nucleic acid sample.

19. The method of claim 18 wherein one of said allelic variants is preferentially extracted from said original nucleic acid sample by a solid phase extraction technique that employs an allele-specific hybridization probe that that is fully complementary to a sequence of one allele of a heterozygous SNP site that is located on said chromosome or chromosomal fragment, wherein said allele specific hybridization probe is attached to a solid support or to a first binding molecule that is capable of binding to a second binding molecule that is attached to a solid support.

20. The method of claim 19 wherein said allele-specific hybridization probe is an oligonucleotide that is attached to a first binding molecule, and said solid phase extraction technique also employs a competitor oligonucleotide that hybridizes to the other allele of the heterozygous SNP site and that is not attached to the first binding molecule

21. The method of claim 18 wherein the genotypes of the chromosomes or chromosomal fragments are determined before one allelic variant of the chromosomes or chromosomal fragments is extracted from the original nucleic acid sample.

22. The method of claim 18 wherein the amount of the enriched allelic variant in the enriched nucleic acid fraction is from 3 to 10 times greater than the amount of the non-enriched allelic variant in the nucleic acid sample.
